(19) United States
(12) Patent Application Publication
(10) Pub. No.: US 2019/0002978 A1
(43) Pub. Date: Jan. 3, 2019

(54) PREGNANCY ASSOCIATED GLYCOPROTEIN (PAG) GENES AS MARKERS OF BULL FERTILITY

(71) Applicant: UNIVERSITY OF TENNESSEE RESEARCH FOUNDATION, Knoxville, TN (US)

(72) Inventor: Ky G. Pohler, Knoxville, TN (US)

(21) Appl. No.: 16/069,403

(22) PCT Filed: Jan. 11, 2017
(86) PCT No.: PCT/US17/12960

§ 371 Date: Jul. 11, 2018

Related U.S. Application Data
(60) Provisional application No. 62/277,180, filed on Jan. 11, 2016.

(51) Int. Cl. C12Q 1/6876

(52) U.S. Cl. CPC C12Q 1/6876; C12Q 2600/158; C12Q 2600/124

(57) ABSTRACT

A method for selecting for enhanced fertility of a male mammal includes obtaining one or more samples of a cell or tissue from a plurality of male mammals and quantifying one or both of a blood pregnancy-associated glycoprotein (PAG) concentration or a PAG genomic DNA in the one or more samples of a cell or tissue. Male mammals of the plurality of male mammals exhibiting a highest circulating PAG and/or a highest PAG genomic DNA are selected. Cells/tissues from the selected male mammals may be utilized in a reproductive procedure. Kits for accomplishing the methods are provided.

This utility patent application claims the benefit of priority in U.S. provisional patent application Ser. No. 62/277,180 filed on Jan. 11, 2016, the entire disclosure of which is incorporated herein by reference.

### TECHNICAL FIELD

The present disclosure relates to markers of male mammal fertility. In particular, the present disclosure relates to the use of pregnancy-associated glycoprotein (PAG) genes as markers for selection of high fertility sires.

### BACKGROUND

In the U.S., the annual cost of reproductive failure to the beef industries is estimated to be $600 million. The exact causes of the preceding reproductive failure include animal management issues, cow infertility, bull infertility, heat stress, and embryonic mortality. Embryonic mortality is thought to be a primary contributor to this loss [1]. During gestation embryonic mortality can occur either early (prior to day 28 of gestation) or late (after day 28 of gestation). Reports of high fertilization rates after a single insemination (˜90% of ovulated oocytes), followed by pregnancy rates of 60 to 70% on day 28 in cows indicate that early embryonic mortality may be 20 to 30% in beef cows [2, 3]. In addition, after day 28 of gestation late embryonic mortality has been reported to vary from 3.2 to 42.7% [4-11]. The large variation in the incidence of late embryonic mortality may be due to differences in cytoplasmic maturity of the oocyte at ovulation, inadequate preovulatory concentrations of estradiol, reduced postovulatory luteal progesterone secretion, inadequate uterine environment, placental insufficiency, and(or) the source of embryos (in vivo fertilized, in vitro fertilized, or cloned by somatic cell nuclear transfer). Cytoplasmic maturity of the oocyte, source of embryos, and placenta sufficiency may affect placental function; whereas, preovulatory estradiol, luteal progesterone secretion, and inadequate dialogue between the embryo and maternal environment may affect endometrial function [12-16].

Significant effort has been directed towards understanding the factors causing early embryonic mortality; however, relatively little is known about the causes or mechanisms associated with late embryonic mortality, much of which occurs around the time of placentome formation (days 35 to 40 of gestation). Although the incidence of late embryonic mortality is normally less than that of early embryonic mortality, the economic consequences of late embryonic mortality can be significant since late embryonic mortality can cause a prolonged delay in conception date and increases cows culled at the end of the breeding season [7]. Previously it has been shown that bovine PAGs (bPAGs) may serve as a marker of late embryonic mortality in beef and dairy cattle [17-20]. Likewise, significant research effort has been directed to conventional markers of bull fertility, i.e. sperm health, motility, quantity, etc. However, to the present investigator's knowledge, no consideration has been given to the potential relationship between paternal PAGs and fertility, i.e. the likelihood of embryo survival according to identity of sire.

### SUMMARY

In accordance with the foregoing need identified in the art, in one aspect methods are provided for selecting for enhanced fertility of a male mammal. The methods include steps of obtaining one or more samples of a cell or tissue from a plurality of male mammals and quantifying one or both of a blood pregnancy-associated glycoprotein (PAG) concentration or a PAG genomic DNA in the one or more samples of a cell or tissue. One or more male mammals of the plurality of male mammals exhibiting a highest circulating PAG and/or a highest PAG genomic DNA are selected. The method may further include using a cell or tissue obtained from the selected one or more male mammals exhibiting the highest circulating PAG or the highest PAG genomic DNA in a reproductive procedure. The male mammals may be bovine animals.

In embodiments, the one or more samples of a cell or tissue from the plurality of male mammals are comprised in one or more blood samples. The reproductive procedure may be one or more of a natural insemination procedure, an artificial insemination procedure, an in vitro fertilization procedure, and a cloning procedure.

In embodiments, the method includes quantifying the blood circulating PAG by an immunoassay. In alternative embodiments, the method includes quantifying the PAG genomic DNA by quantifying one or more PAG genes selected from the group consisting of a PAG 7 gene, a PAG 8 gene, a PAG 11 gene, a PAG 20 gene, and a PAG 21 gene.

In embodiments, the PAG 7 gene comprises one or both of SEQ ID NO: 15 and SEQ ID NO: 16 and is quantified in genomic DNA by PCR using primers selected from one or both of SEQ ID NO: 1 and SEQ ID NO: 2. In embodiments, the PAG 8 gene comprises one or more of SEQ ID NO: 17, SEQ ID NO: 18, SEQ ID NO: 19, and SEQ ID NO: 20 and is quantified in genomic DNA by PCR using one or more primers selected from SEQ ID NO: 3, SEQ ID NO: 4, SEQ ID NO: 5, and SEQ ID NO: 6. In embodiments, the PAG 11 gene comprises one or both of SEQ ID NO: 21 and SEQ ID NO: 22 and is quantified in genomic DNA by PCR using primers selected from one or both of SEQ ID NO: 7 and SEQ ID NO: 8. In embodiments, the PAG 20 gene comprises one or both of SEQ ID NO: 23 and SEQ ID NO: 24 and is quantified in genomic DNA by PCR using primers selected from one or both of SEQ ID NO: 9 and SEQ ID NO: 10. In embodiments, the PAG 21 gene comprises one or more of SEQ ID NO: 25, SEQ ID NO: 26, SEQ ID NO: 27, and SEQ ID NO: 28 and is quantified in genomic DNA by PCR using primers selected from one or more of SEQ ID NO: 11, SEQ ID NO: 12, SEQ ID NO: 13, and SEQ ID NO: 14.

In another aspect, a kit for determining enhanced fertility of a male mammal is provided, comprising reagents for quantifying a blood pregnancy-associated glycoprotein (PAG) concentration and/or quantifying a PAG genomic DNA in one or more samples of a cell or tissue obtained from a plurality of male mammals. Optionally, one or more of: equipment for obtaining the one or more samples of a cell or tissue, additional reagents, and instructions for use of said reagents for quantifying may be provided. The male mammals may be bovine animals.

In embodiments, the kits include reagents for quantifying the blood circulating PAG by an immunoassay. In other embodiments, the kits include reagents for quantifying PAG genes selected from one or more of a PAG 7 gene, a PAG 8 gene, a PAG 11 gene, a PAG 20 gene, and a PAG 21 gene. The PAG genes may be substantially as described above. Likewise, the kits may include primers for quantifying PAG genes, substantially as described above.

In the following description, there are shown and described embodiments of the disclosed methods and kits for selecting for enhanced male mammal fertility. As it should be realized, the devices are capable of other, different embodiments and their several details are capable of modification in various, obvious aspects all without departing from the devices and methods as set forth and described in the following claims. Accordingly, the drawings and descriptions should be regarded as illustrative in nature and not as restrictive.

### BRIEF DESCRIPTION OF THE DRAWINGS

The accompanying drawing figures incorporated herein and forming a part of the specification, illustrate several aspects of the disclosure, and together with the description serve to explain certain principles thereof. In the drawings:

FIG. 1 shows Serum concentrations of bovine pregnancy associated glycoproteins (bPAGs) in pregnant Nelore cows (mean±SEM; n=22) from day 0 to 30 of gestation in Exp. 1. First significant increase (P<0.0001) in circulating bPAGs concentrations occurred on day 24 of gestation;

FIG. 2 shows Serum concentrations of bPAGs (mean±SEM) in postpartum pregnant Nelore beef cows (primiparous n=116; multiparous n=687) which received TAI on day 0 and had a viable embryo on day 28 of gestation. Primiparous Nelore beef cows had increased (P<0.05) circulating concentrations of bPAGs on day 28 compared to multiparous cows independent of body weight;

FIG. 3 shows Serum concentrations of bPAGs (mean±SEM) in postpartum Nelore beef cows which received TAI on day 0 and had a viable embryo on day 28 of gestation (n=803) and either maintained (embryonic survival; n=714) or experienced embryonic mortality (n=89) by day 100. Nelore cows that experienced late embryonic mortality by day 100 of gestation had decreased (P<0.05) circulating concentrations of bPAGs on day 28 compared to cows that maintained an embryo until day 100;

FIG. 4 shows Probability of pregnancy maintenance following TAI between day 28 to 100 of gestation based on day 28 serum concentrations of bPAGs (n=803). Increased serum concentrations of bPAGs on day 28 significantly increased (P<0.05) the probability of pregnancy maintenance until day 100 of gestation in Nelore beef cows following TAI;

FIG. 5 shows Receiver operating curve (ROC) utilizing day 28 circulating concentrations of bPAGs to model embryonic mortality between days 28 to 100 of gestation in Nelore beef cows following TAI. A serum concentration of bPAG below 0.72 ng/mL resulted in a 95% confidence that embryonic mortality would occur between days 28 to 100 of gestation with an area under the curve of 81.6 (P<0.05). The ROC curve graphically display the relationship between true positive rate (Sensitivity) and false positive rate (1-Specificity) when an increasing cutoff for the bPAG test was applied. When the true positive rate and the false positive rate both decrease as the cutoff value is increased this results in a diagonal line through the center meaning the test is not predictive (50:50 probability). However, when the line is deflected to the left of center the test is useful since it has a relatively high true positive rate and a low false positive rate at a specific cutoff;

FIG. 6 shows Serum concentrations of bPAGs on day 28 of gestation from cows with pregnancies sired by sires 1 to 8. Although there was variation in pregnancy rate to TAI among sires (44 to 64%), there was no linear relationship between pregnancy rate by sire and circulating concentrations of bPAGs. However, there were significant differences in circulating concentrations of bPAGs among sires;

FIG. 7 shows Serum concentrations of bPAGs on day 28 of gestation between sires that resulted in high embryonic loss and sires that resulted in low embryonic loss. After removing all cows that lost a pregnancy after day 28 from the data set, the sires with the highest incidence of late embryonic mortality also were the sires with pregnancies that produced significantly (P<0.05) lower maternal circulating concentrations of bPAGs on day 28 of gestation compared to the remaining sires that had pregnancies having low embryonic mortality

FIG. 8 shows Serum concentrations of bPAGs (mean±SEM) in postpartum primaparous Nelore beef cows which received TAI on day 0 and had a viable embryo on day 28 of gestation (n=303) and either maintained (embryonic survival; n=285) or experienced embryonic mortality (n=18). Nelore beef cows that experienced late embryonic mortality by day 100 of gestation had decreased (P<0.05) circulating concentrations of bPAGs on day 28 compared to cows that maintained an embryo until day 100);

FIG. 9 shows serum concentrations of bPAGs (mean±SEM) in postpartum primaparous Nelore beef cows which received TAI on day 0 and had a viable embryo on day 28 with different levels of Estrotect Patch activation on Day 0 (0, lost patch; 1, <25% activated; 2, <50% activated; 3, <75 activated; 4, >75% activated). As intensity of estrus expression increased, as determined by Estrotech patch score at TAI, there was s significant increase in circulating bPAG concentrations on Day 28.

FIG. 10 shows day 30 maternal circulating PAG by breed of bull;

FIG. 11 shows day 30 maternal circulating PAG for multiple bulls of the same breed;

FIG. 12 shows a plot of circulating maternal PAG and pregnancy rate according to fertility of bull;

FIG. 13 shows a comparison of circulating maternal PAG when bulls are grouped according to high and low embryonic loss;

FIG. 14 shows sequences used for generation of various primers for quantifying genomic PAGs;

FIG. 15 shows variance in circulating maternal PAG for sires of differing fertility;

FIG. 16 shows embryonic loss variance among bulls of differing fertility;

FIG. 17A shows variance in PAG 20 gene levels for bulls grouped by pregnancy rate;

FIG. 17B shows variance in PAG 20 gene levels for bulls grouped by embryonic loss rate;

FIG. 18 shows variance in PAG 7 gene levels for bulls grouped by embryonic loss rate

FIG. 19 shows variance in PAG 8 gene levels for bulls grouped by embryonic loss rate;

FIG. 20 shows variance in PAG 11 gene levels for bulls grouped by embryonic loss rate; and

FIG. 21 shows variance in PAG 21 gene levels for bulls grouped by embryonic loss rate.

Reference will now be made in detail to embodiments of the disclosed subject matter, examples of which are illustrated in the accompanying drawing figures.

### DETAILED DESCRIPTION

Any citations, gene sequences, accession numbers, and reference sequences included or referred to in this application form a part of the disclosure and are incorporated herein in their entirety by reference. It will be appreciated that the embodiments shown and described in this patent application are an illustration of one of the modes best suited to carry out the invention. The invention is capable of other different embodiments, and its several details are capable of modification in various, obvious aspects all without departing from the invention. Accordingly, the drawings and descriptions provided herein will be regarded as illustrative in nature and not as restrictive. Various embodiments of the methods and compositions of the present disclosure will now be described by way of the following Examples.

### Materials and Methods—Examples 1-3

Experiments were conducted in a commercial beef farm located in Mato Grosso, Brazil and were conducted in accordance with the Guide for the Care and Use of Agricultural Animals in Agricultural Research and Teaching (FASS, 1999). In all experiments cows were maintained on pastures, specifically Brachiaria brizantha with water and mineral salt ad libitum. Cows used in all three experiments below were at least 25 days postpartum when the estrus synchronization protocol began. All cows received an intravaginal progesterone (P4) insert containing 1.9 g of P4 (CIDR, Zoetis, São Paulo, Brazil), and 2.0 mg (i.m.) estradiol benzoate (EB, 2.0 mL of Estrogin, Farmavet, São Paulo, S P, Brazil) on day −11, CIDR withdrawal, 25 mg (i.m.) dinoprost tromethamine (PGF; 5.0 mL of Lutalyse, Zoetis, Brazil), 300 i.u. of equine chronic gonadotropin and 1.0 mg (i.m.) of estradiol cypionate (0.5 mL of E.C.P., Zoetis, Brazil) on day −2, and fixed-time AI (TAI) on day 0. Following TAI, all cows were diagnosed for pregnancy at day 28-30 of gestation. Pregnancy determination was based on the presence of a viable embryo (presence of a heart beat) as detected by ultrasound (US) scan. Following confirmation of pregnancy a blood sample was collected for quantification of bPAG. All cows were then confirmed pregnant at day 100 of gestation.

Animals/Treatment/Procedures

### Experiment 1

Postpartum Nelore beef cows (n=56) were artificially inseminated at a fixed time following synchronization of ovulation (day 0) by using the protocol described above. Prior to TAI on day 0, the size of the ovulatory follicle was also determined by ultrasound. Serum samples were collected on days 0, 21, 24, 27, and 30. All samples were harvested by venipuncture into a 10-ml vacutainer tube and allowed to clot at room temperature for 1 hour before being placed in a 4° C. refrigerator for 24 hours. Following centrifugation, serum was collected and stored at −20° C. until measurement of bPAGs was performed.

### Experiment 2

Synchronization of estrus and TAI in postpartum Nelore beef cows (n=1460) was conducted as described. In this experiment there were both primiparous (n=240) and multiparous cows (n=1220). A subset of the cows (n=720) were artificially inseminated at a fixed time to eight Angus sires (n=90 cows per sire) to assess the effects of sire on pregnancy rate to TAI and day 28 bPAG concentrations. All other cows were randomly assigned to be inseminated with semen from Angus sires of proven fertility. Serum samples were collected from all cows on day 28 post insemination as explained in experiment 1.

### Experiment 3

Ovulation was synchronized in primiparous postpartum Nelore beef cows (n=689) as described above and received TAI on day 0. Cows were inseminated randomly to Angus sires of proven fertility. In addition, Estrotect Heat Detector patches were scored on a scale of 0 to 4 (0=lost patch, 1<25% activated, 2<50% activated, Serum samples were collected from all cows on day 28 post insemination as explained in experiment 1.

Assays

Serum concentrations of progesterone were quantified by RIA with Coat-a-Count RIA kit (Diagnostic Products Corporation, Los Angeles, Calif.) as described previously [21, 22]. Intra-assay coefficient of variation was 5% and the assay sensitivity was 0.08 ng/mL for the progesterone RIA. Serum concentrations of bPAGs were determined by a monoclonal-based bPAG ELISA similar to that described by Green et al., [23] and used previously to monitor bPAGs [19, 24]. Each assay was run with a standard curve, a sample from a pregnant cow from day 60 of gestation and a pooled sample from a non-pregnant cow.

Statistical Analysis

One-way ANOVA (SAS 9.4) was used to test differences among day 28 circulating concentrations of bPAGs for beef cows undergoing TAI that maintained pregnancy and those that established a pregnancy, but did not maintain it. The LOGISTIC procedure in SAS (9.4) was used to determine the probability of pregnancy maintenance based on a single day 28 serum concentration of bPAG. Receiver operating charactertisc curves (ROC) were generated with the MedCal software package, setting embryonic mortality as the ‘true positive’. Following the generation of an ROC, the resulting true positive and false positives were subjected to positive and negative predictive value analysis to determine a concentration of bPAGs on day 28, below which 95% of cows would experience embryonic loss by day 100. Analysis of breakpoints was conducted by using PROC NLIM [25] in SAS, and was used to determine the first significant change in the slope of the line.

### Results—Example 1

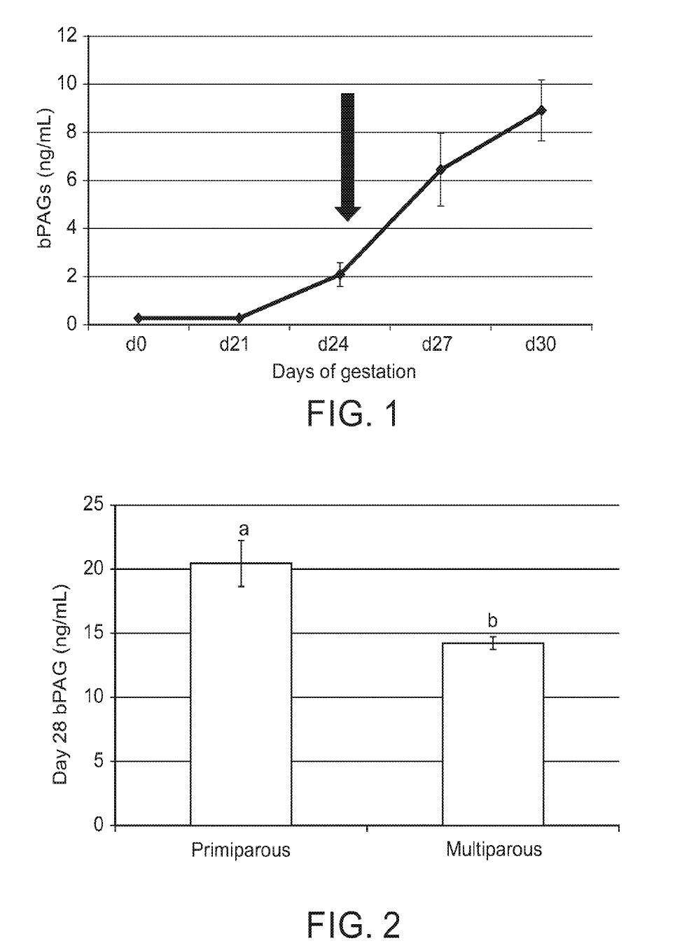

Overall pregnancy rate to TAI was 39% (n=22) and based on break point analysis the first significant increase (FIG. 1; P<0.0001) in bPAG concentration occurred at day 24 of gestation. In addition, there was no significant effect of ovulatory follicle size (P=0.44) at day 0 on circulating concentrations of bPAGs at d 28 or any relationship between circulating P4 (P=0.37) and bPAG concentrations on day 28.

### Results—Example 2

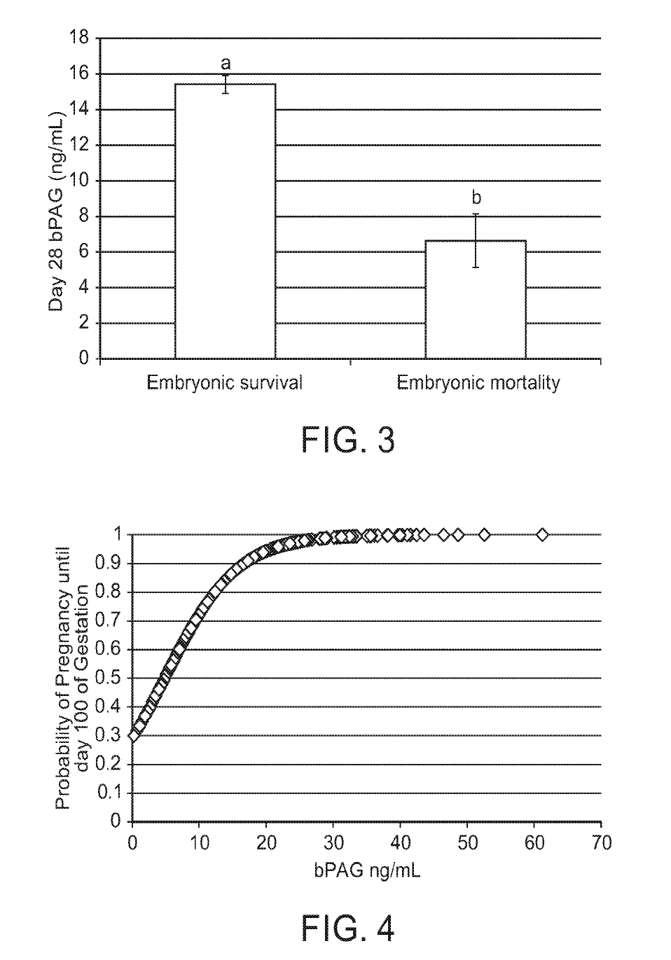

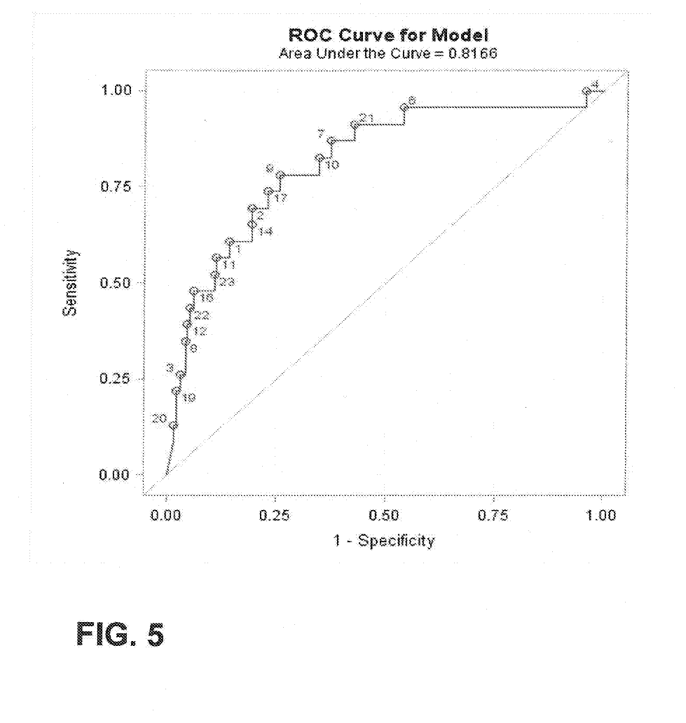

The day 28 pregnancy rate was 55% (n=803); pregnancy was confirmed based on a viable fetal heartbeat visualized by transrectal ultrasonography. The average serum concentration of bPAGs on day 28 was 15.11±9.92 ng/ml (mean±SD). Serum concentrations of bPAGs were higher (FIG. 2; P<0.03) in primiparous cows (n=116; 20.45 ng/ml±1.80 ng/ml; mean±SEM) compared to multiparous cows (n=687; 14.23 ng/ml±0.49 ng/ml; mean±SEM). There was no relationship between body weight and bPAG concentrations across all cows tested. In addition, cows that maintained a pregnancy from days 28 to 100 of gestation (n=714) had significantly (FIG. 3; P<0.0001) higher circulating concentrations of bPAGs on day 28 of gestation compared to cows that did not maintain a pregnancy (EM) to day 100 (n=89). When day 28 bPAG concentration was included in a logistic regression model to predict pregnancy maintenance until day 100 of gestation, there was an increase (FIG. 4; P<0.0001) in the probability of maintaining pregnancy to day 100 of gestation as maternal concentrations of bPAGs increased. To conduct a more stringent test of the effectiveness of a single circulating bPAG concentration to predict embryonic survivability/mortality, a receiver-operating characteristic (ROC; FIG. 5) curve was generated to determine bPAG concentrations on day 28 that should predict embryonic survival or mortality with ≥95% accuracy. Based on positive and negative predicative value analysis, a circulating concentration of bPAGs above 7.9 ng/ml was 95% accurate in predicting embryonic maintenance (to day 100) and a concentration of bPAGs below 0.72 ng/ml (minimal detectable level 0.28 ng/mL) was 95% accurate in predicting EM (between day 28-day 100) at day 28 of gestation. In addition, P4 concentrations were not significantly associated with bPAG in circulation or predictive of late embryonic mortality.

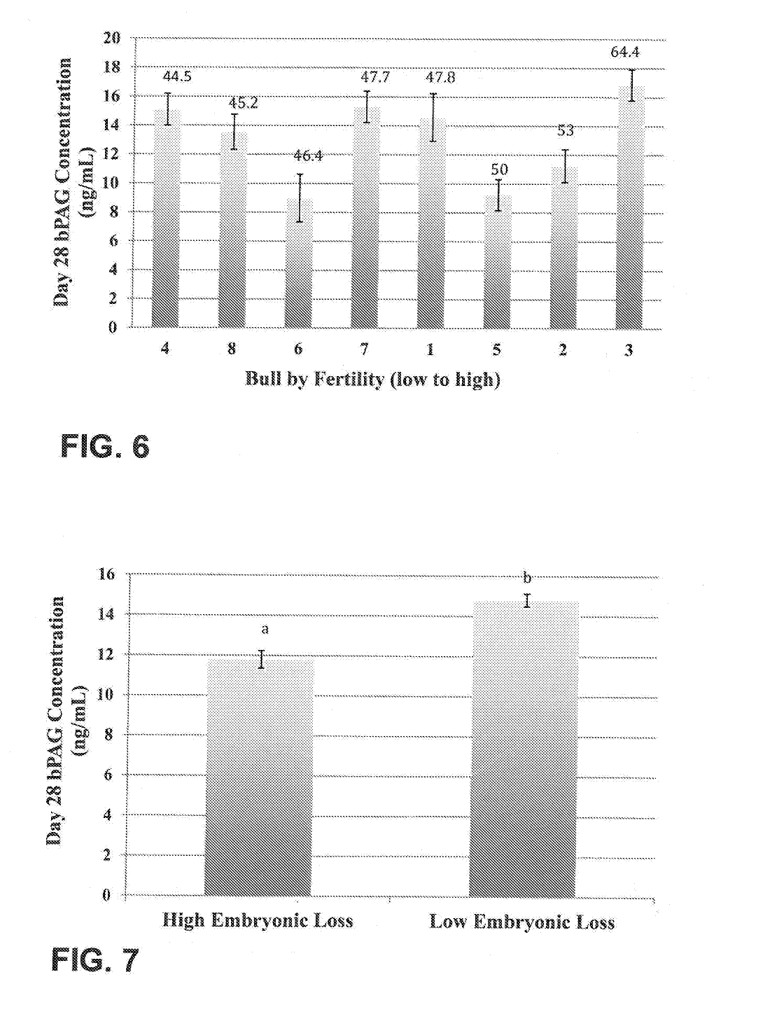

A subset of cows in this experiment (n=720; pregnant at d 30 n=396) were evaluated for a sire effect on bPAG concentrations on day 30 of gestation. Although there was variation in conception rate to TAI among sires (44 to 64%), there was no linear relationship between pregnancy rate by sire and circulating concentrations of bPAGs (FIG. 6). However, there were significant differences in circulating concentrations of bPAGs among sires. There were 39 cows that established a pregnancy to TAI and had a viable embryo on day 28 of gestation but failed to maintain pregnancy to day 100 of gestation. Three sires in this experiment accounted for over 70% of the late embryonic mortality. After removing all cows that lost a pregnancy after day 28, the three sires with the highest incidence of late embryonic mortality were the sires whose pregnancies produced significantly (P<0.05; FIG. 7) lower maternal circulating concentrations of bPAGs on day 28 of gestation compared to the five remaining sires with pregnancies that experienced lower embryonic mortality.

### Results—Example 3

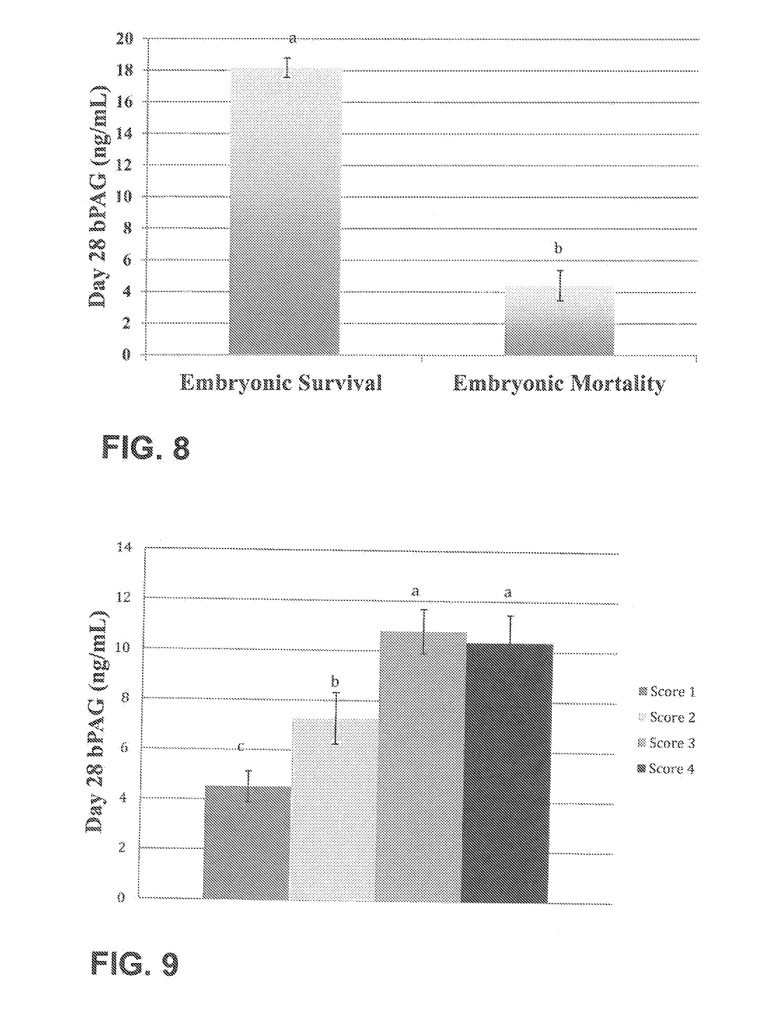

Primiparous Nelore beef cows underwent pregnancy diagnosis at day 30 of gestation and the average bPAG concentrations for all pregnant cows was 17.42±10.80 ng/mL (n=303; mean±SD). As observed in experiment 2, there was a significant difference in day 30 bPAG concentrations between cows that successfully established and maintained (n=285; 18.18±0.61 ng/mL; mean±SEM) a pregnancy compared to those that established and failed to maintain a pregnancy (n=18; 4.41±0.95 ng/mL; mean±SEM P<0.05; FIG. 8). In addition, the cutoff concentration of bPAGs developed in experiment 2 (0.72 ng/mL) was 95% accurate in predicting cows that would experience embryonic mortality in experiment 3. No cows that actually maintained pregnancy fell below the cutoff value for prediction of embryonic mortality. However, on the prediction of embryonic survivability, we were not as successful. Based on the cutoff value of 7.9 ng/mL from experiment 2, 5 cows that ended up undergoing embryonic mortality would have been predicted to maintain.

### DISCUSSION

Bovine pregnancy associated glycoproteins (bPAGs) are detected in the maternal circulation beginning around day 24 to 26 after insemination [19, 23], and may serve as a marker for placental function [18]. Bovine PAGs were identified by multiple groups following their purification from placental extracts as well as their detection in the maternal circulation [26-30]. Since that time the focus of bPAG research has centered on development of accurate assays for detecting bPAGs in blood and milk for the purpose of pregnancy diagnosis. In the present study, bPAGs were 96% accurate in diagnosing pregnancy in Bos indicus (Nelore) beef cows suggesting that bPAGs can work in crosses of subspecies (Bos taurus/Bos indicus). Currently, there are 3 commercially based assay platforms that utilize bPAGs for diagnosis of pregnancy in cattle either by blood or milk [31], and all have been demonstrated to accurately diagnosis pregnancy. The assay platform utilized in these experiments represents the commercially available test, however, provides quantitative based measurements of PAG.

Reports of late embryonic/fetal mortality in cattle range in the literature from ˜3-40% depending on the cow type and location [4-9, 11]. In the current study, the incidence of late embryonic mortality was ˜11 and 6% for experiments 2 and 3, respectively. The increased incidence of late embryonic mortality in experiment 2 was not surprising since multiparous cows have been shown to have increased late embryonic mortality compared to younger cattle [6, 32].

The exact mechanisms that lead to late embryonic mortality have been poorly characterized due in part to the need for a model to identify those cattle that will maintain or not maintain a pregnancy. Multiple reports have demonstrated that circulating concentrations of bPAGs may be associated with late embryonic mortality in cattle [17-19, 33, 34]; however, other reports have demonstrated no such association [35]. In Bos taurus beef cattle (Angus, Hereford, etc.) undergoing both TAI and embryo transfer (ET), bPAG concentrations at day 28-30 of gestation have been shown to be significantly increased in cows establishing and successfully maintaining a pregnancy until day 60-72 of gestation compared to those that establish, but do not maintain, a pregnancy during that time period [18, 19]. Furthermore, data in dairy cows showed that an increase in circulating bPAG concentrations at day 28 to 30 was associated with pregnancy success [17, 20, 34, 36]. Similar results have been shown in sheep pregnancy too [37]. However, there is conflicting data to suggest that bPAGs around day 30 of gestation are not predictive of late embryonic mortality in high producing dairy cattle [35]. In both of the beef studies above, along with the dairy studies published by Thompson et al., [20] and Pohler et al., [34] the assay platforms utilized in those experiments were very similar in that they used the exact same monoclonal based sandwich ELISA validated by Green et al., [23]. These data suggest that the bPAG assay platform has the potential to have a major impact on the usefulness of bPAG measurements for diagnosing pregnancy and predicting late embryonic mortality.

In the present study, we aimed to develop a cutoff model by using the same sandwich ELISA platform that has shown utility in previous reports [19, 23, 24]. In this experiment similar results were observed. Cows undergoing late embryonic mortality between days 28-100 of gestation had significantly decreased circulating concentrations of bPAGs at day 28 of gestation compared to cows that successfully maintain a pregnancy. In addition, based on ROC curves and positive/negative predictive value analysis we were able to determine a circulating concentration of bPAG at day 28 of gestation that was predictive of embryonic mortality or survivability. Previous studies have shown associations between bPAG concentrations and late embryonic mortality; however, this model has allowed for prediction of pregnancy success during day 28 to 100 of gestation. In experiment 3, this model was tested in a separate set of cows to validate its ability to detect late embryonic mortality. In primiparous Nelore beef cows the model was 95% accurate in predicting late embryonic mortality below 0.72 ng/ml; however, it was not 95% accurate in predicting embryonic maintenance. One possible explanation for this is that primiparous cows were shown in experiment two to have significantly increased circulating concentrations of bPAGs at day 28 of gestation which could explain the higher cutoff value for predicting embryonic survivability even though primiparous cows were included in the original model construction in experiment 2. Another possible explanation is that bPAGs have been shown to only be predictive of pregnancy loss between days 28-40 of gestation [19] therefore evaluating embryonic loss from day 28 until day 100 of gestation may encompass too much time. Indeed, Pohler et al., [34], suggest that bPAGs are really only predictive of embryonic mortality between day 28-45 of gestation and does not take into account the possibility of ovarian failure or other types of pregnancy loss they may occur after day 45 of gestation.

Circulating concentrations of bPAGs have been reported to increase in maternal circulation around day 24 of gestation until about day 36 in Bos taurus cattle and subsequently decrease until about day 60 of gestation; circulating bPAGs then begin to increase again between days 60-90 and they steadily rise throughout gestation until reaching a peak around the time of parturition [19]. In experiment 1, a similar rise in bPAGs early in gestation was observed in the Bos indicus cows used in this study. Although there is a large transient rise in bPAGs during early gestation, no clear function has been identified for these proteins; however, there have been many correlations reported with circulating concentrations of bPAGs. Pregnancy status and stage, breed, parity of dam, fetal sex and number, fetal birth weight, placental weight, sire, and many more have been shown to be associated to some degree with bPAG concentrations [19, 38-40]. In the current study, circulating concentrations of bPAG at day 28 were influenced by parity of the dam, sire, and breed. In a recent study, Mercadante, Waters [24] reported that cows with Bos indicus genetics (similar to Nelore) had increased circulating concentrations of plasma bPAGs early in gestation. We observed similar results in the present study based on comparison of the current data to bPAG data collected from similar stages of gestation in Bos taurus cattle. The exact cause of this increased bPAG concentration early in gestation is not clear; however, Mercadante, Waters [24] also reported differences in fetal size and growth rate. Interestingly, parity of the cow also had a large effect on circulating bPAG concentration on day 28 of gestation independent of overall body weight, which is a good measure of overall blood volume. Similar results have been shown by Kill et al., [32], which reported that Bos taurus heifers had significantly higher circulating bPAG concentrations compared to mature cows. These data suggest that it is not a simple blood dilution effect and that some other mechanism is taking place in these younger animals. Potential explanations could be the half-life of bPAGs in those individual types of animals, the ability of BNC to secrete products into the maternal circulation or maybe even the function or role that bPAGs are playing in these younger animals.

Multiple factors such as parity status and sire were shown to effect circulating concentrations of bPAG at day 28. Little is known regarding sire effects on bPAG concentrations early in gestation; however, based on the large influence that the sire plays in placental development there was interest in examining this relationship. Overall, we saw no relationship between circulating concentrations of bPAGs and sire fertility, but there was a large amount of variation across sires and bPAG production. In addition, of eight sires tested three accounted for 70% of the late embryonic mortality reported in the subset of cows in experiment 2. Surprisingly, after removing from the analysis all the cows that underwent late embryonic mortality after day 28, those three sires exhibited significantly decreased circulating concentrations of bPAG compared to the other five sires in the study. Taken together these data suggested that the sire could influence BNC products, such as bPAGs. Indeed, there was potential that bPAG may serve as a novel tool for identifying low fertility sires.

### Example 4

To further explore this relationship, 1228 multiparous Nelore cows were artificially inseminated. A subset of the cows were inseminated with Nelore vs Angus bulls to determine the potential for breed effect on PAG. Another subset of cows were inseminated with semen collected from 6 different Angus bulls in order to evaluate bull fertility on PAG early in gestation. Blood collection and measurement of circulating bPAG were as described above.

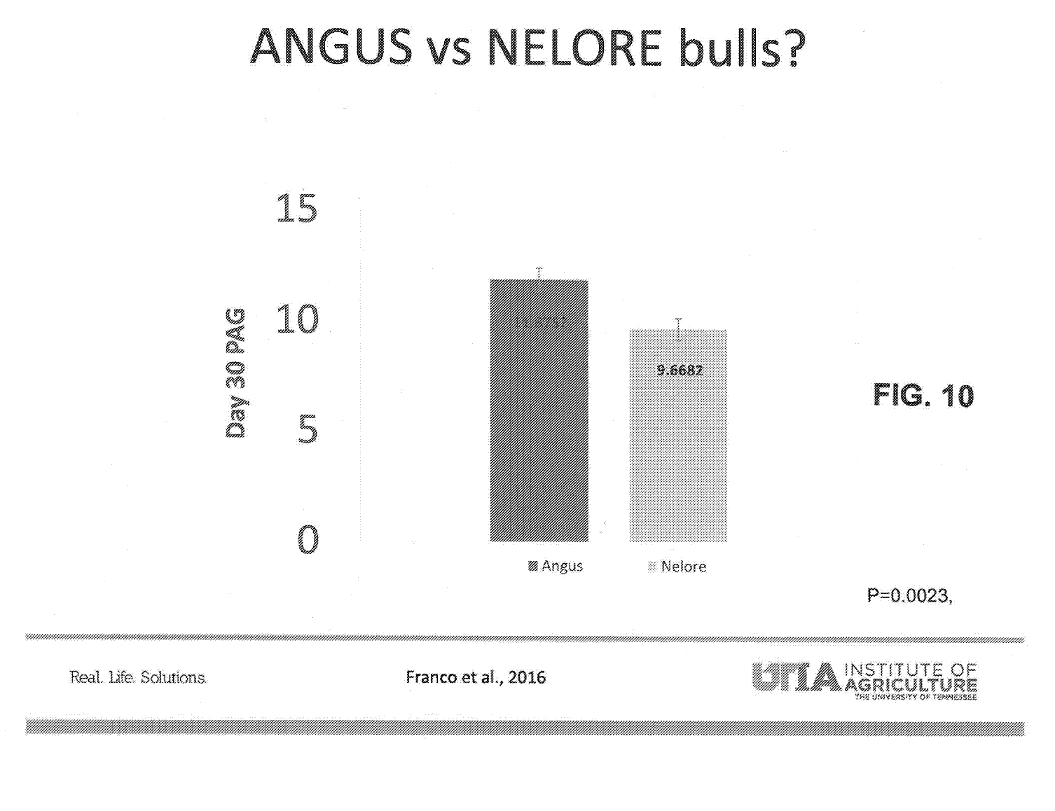

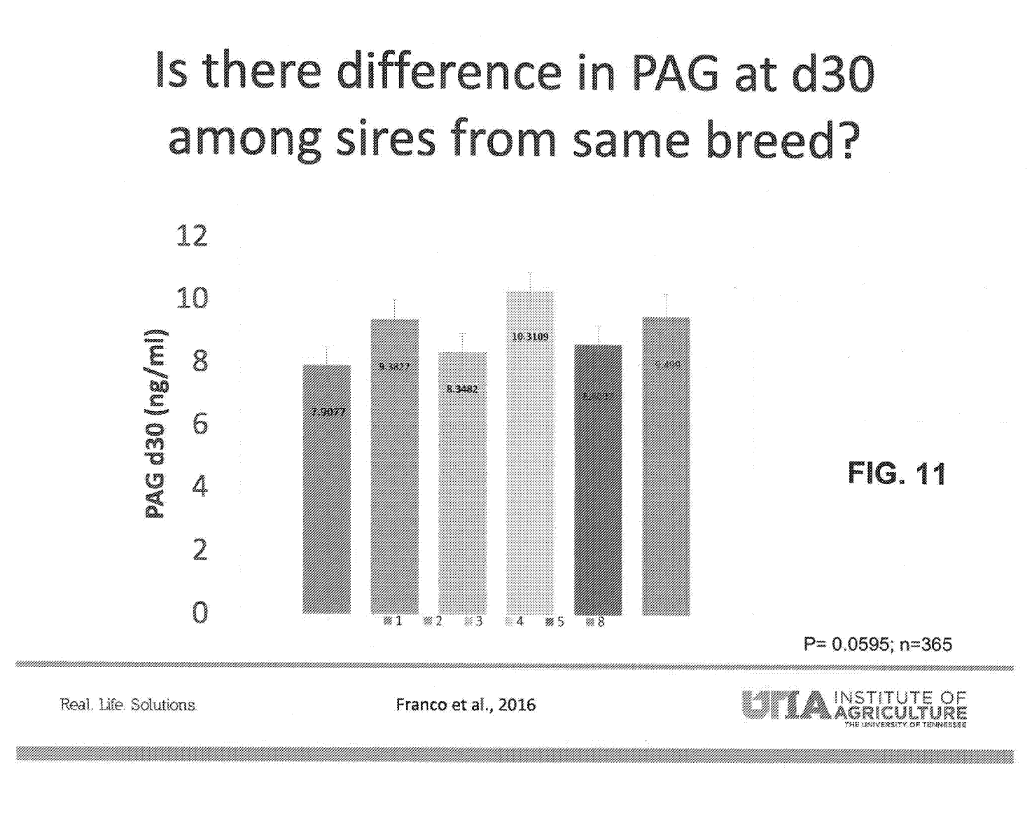

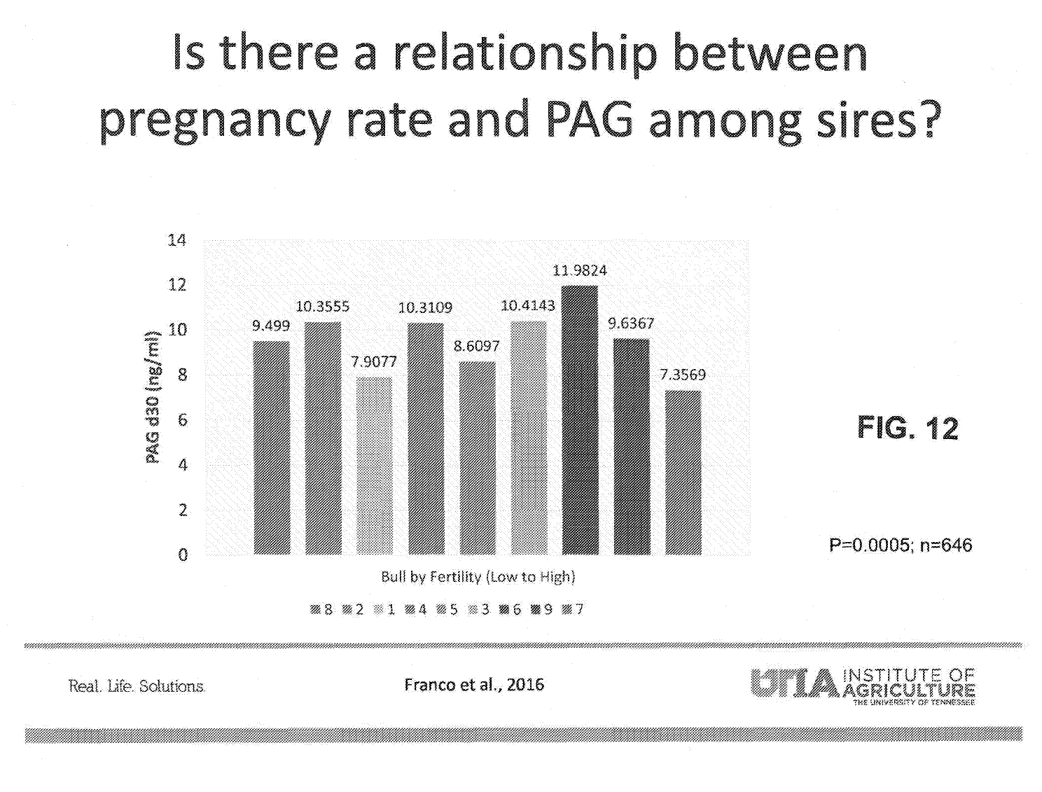

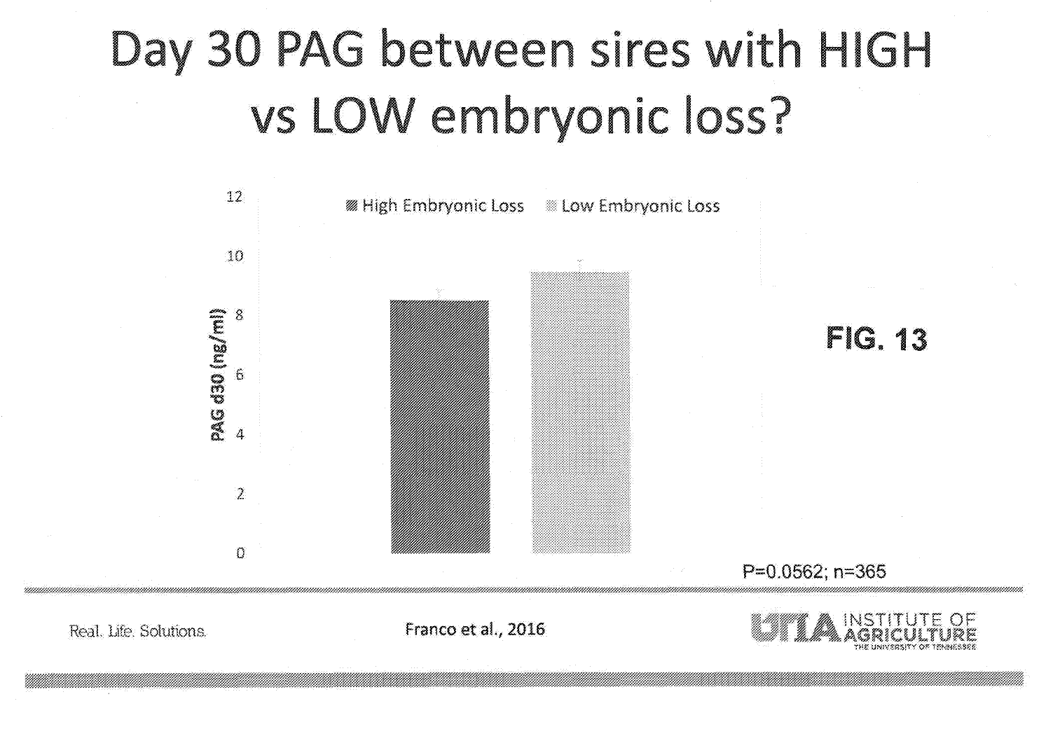

A slightly higher maternal day 30 circulating PAG (ng/ml) was found in Angus bulls vs Nelore bulls (FIG. 10). When data from 6 different Angus bulls were analyzed, maternal day 30 circulating PAG (ng/ml) varied according to sire (FIG. 11) Likewise, no clear relationship was found between maternal day 30 circulating PAG (ng/ml) and sire (FIG. 12). However, when maternal day 30 circulating PAG (ng/ml) was analyzed between cows with high embryonic loss and low embryonic loss, there appeared to be a sire effect (FIG. 13).

### Example 5

612 Angus cows were artificially inseminated using semen randomly selected from 9 Angus bulls having unknown fertility. The cows were pregnancy diagnosed on day 31 after insemination. Blood samples were collected on days 0 (day of insemination), day 24, and day 31. Quantification of circulating PAG was as described above.

PAG genomic DNA from bulls was quantified by qPCR. 1 ng of DNA was added to Power SYBR Green PCR master mix (Applied Biosytems) and primers (200-800 nM final concentration according to gene of interest) in duplicate. Each experiment included no-template controls (nuclease-free water) and a pool of Holstein bull DNA (qPCR calibrator) in duplicate for each gene. A “housekeeper” control of SRY3 was also assayed for normalization (SRY3 exhibited similar levels across bulls). Samples were assayed for 45 cycles at an annealing temperature of 58° C. followed by a dissociation curve. Data were normalized to SRY3 and expressed as a proportion of values for the Holstein poll (“calibrated”) according to MIQE standards (Bustin et al., 2009 The MIQE Guidelines: Minimum Information for Publication of Quantitative Real-time PCR Experiments, Clin. Chem. 55:4, 611-622). The primers used to quantify PAG genes of interest are presented in Table 1. The PAG genes of interest were the PAG 7 gene, the PAG 8 gene, the PAG 11 gene, the PAG 20 gene, and the PAG 21 gene.

TABLE 1
Primers for genomic PAG quantification.

| Gene of | Forward Primer | Reverse Primer |
|---|---|---|
| Interest | (5′-3′) | (5′-3′) |
| PAG 7-3 | GCCACTGCTATACCACCTTT | CCTTAATGATTCTTGAGCCA |
| | A (SEQ ID NO: 1) | GACC (SEQ ID NO: 2) |
| PAG 8-2 | AGACCTGGATCCTGGGTGAT | TGTGTGTTTGTCTGGCCCTG |
| | (SEQ ID NO: 3) | (SEQ ID NO: 4) |
| PAG 8-3 | TTTCAAGGGGGCACAGAGAT | ATTGATTCCTGTAGCAGCCC |
| | (SEQ ID NO: 5) | G (SEQ ID NO: 6) |
| PAG 11* | AGTGATTGGCTGTGAACACG | CACGAGGCACTGAGTAATCG |
| | (SEQ ID NO: 7) | (SEQ ID NO: 8) |
| PAG 20* | CCGGGTCATCAAATATCCAA | GACAACAGAGGGCAGAGCAC |
| | (SEQ ID NO: 9) | (SEQ ID NO: 10) |
| PAG 21-2 | TATACCGCCTTTAAAAAGCA | GTGCCCTAGTGTGAGTGAGT |
| | ACG (SEQ ID NO: 11) | G (SEQ ID NO: 12) |
| PAG 21-3 | GTGTCAGAATACGGGTTTAA | GATGGGGATGGCTTCAGAGA |
| | GGA (SEQ ID NO: 13) | A (SEQ ID NO: 14) |

*These are from Table 1 in Touzard et al, Reproduction 2013 146:347-362. Others were designed using NCBI Primer-BLAST software. Primer sets were chosen NOT to span an intron since looking at genomic DNA rather than transcripts and to yield products ~150 to 200 bp in length (for optimal qPCR performance). The number after the dash (i.e., -2 or -3) is an in-house designation indicating that it was the 2^{nd} or 3^{rd} primer set tried for that gene.

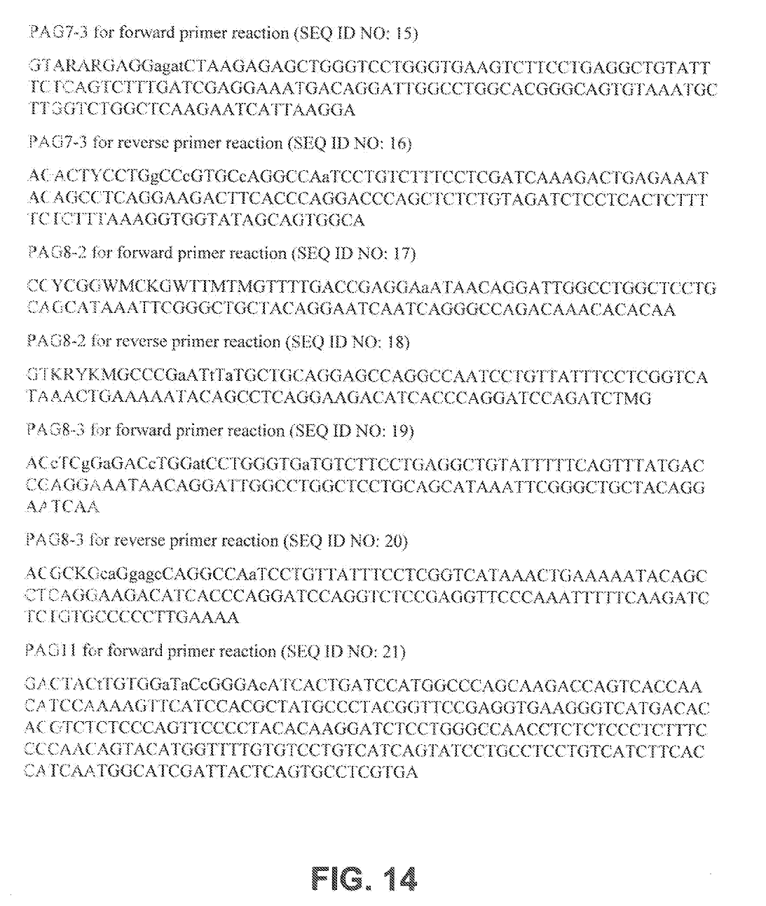

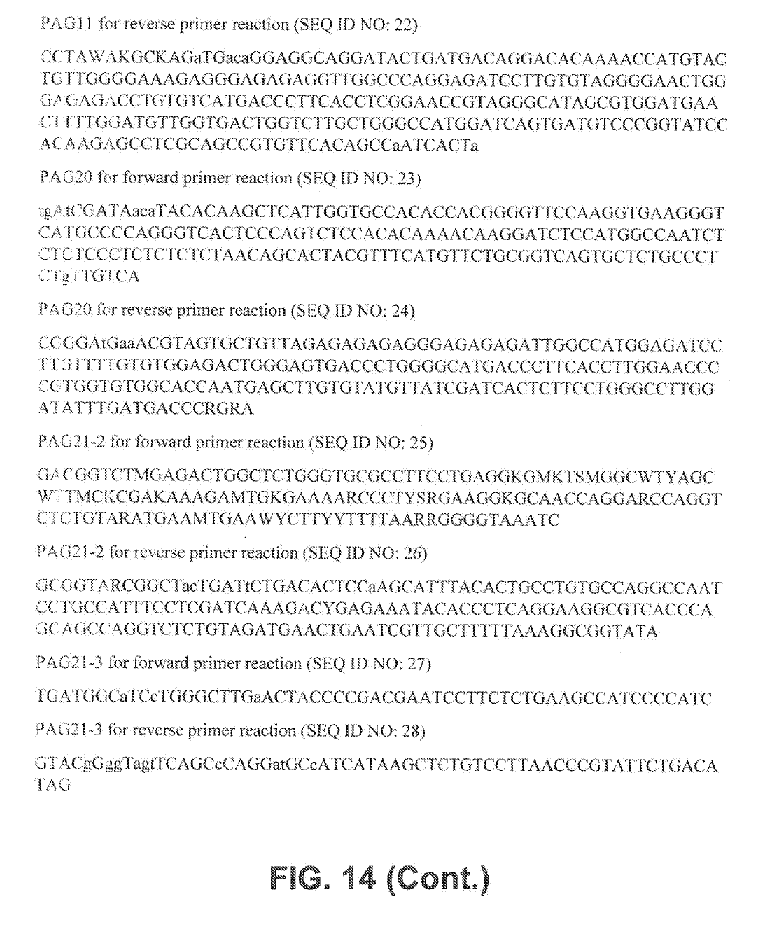

Sequence information (Sanger data) for the PAG genes of interest used to synthesize the primers shown in Table 1 are presented in FIG. 14.

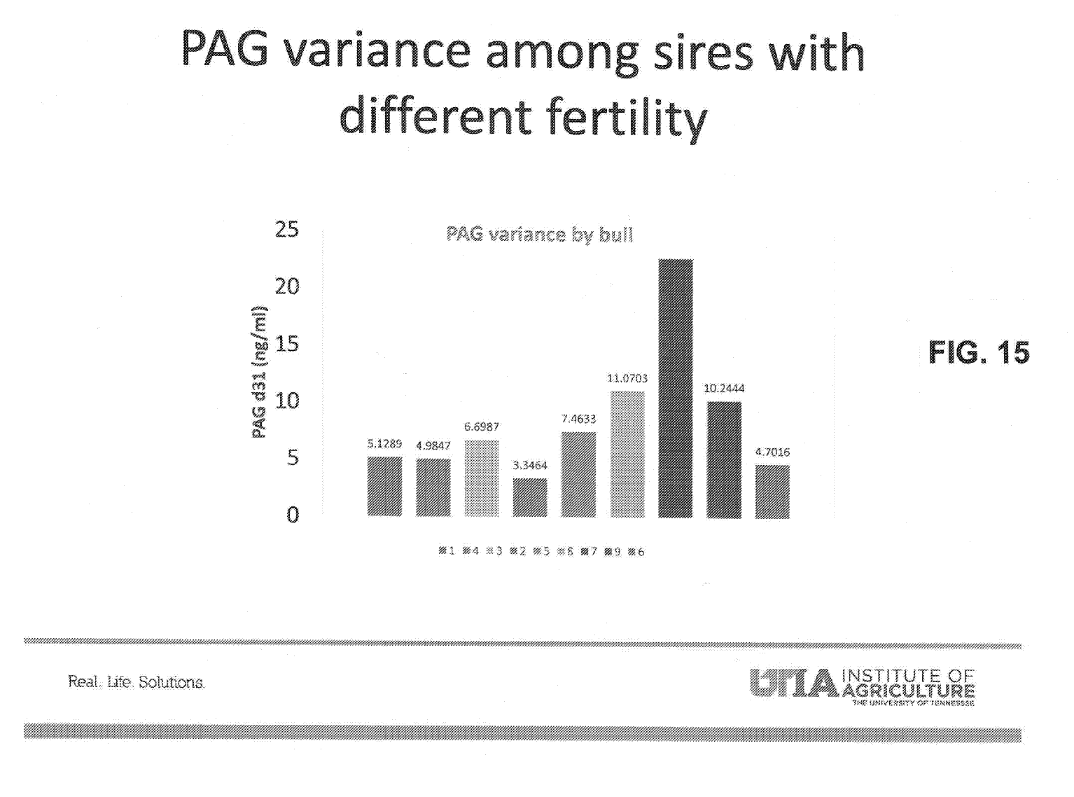

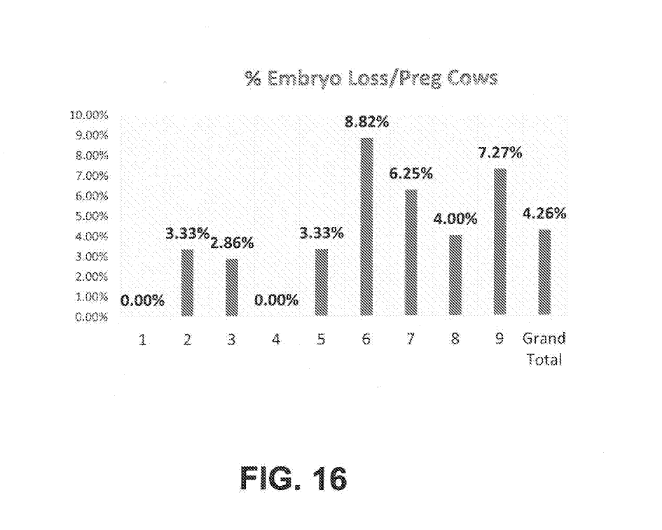

Cow pregnancy rate did not appear significantly affected by bull PAG variance (data not shown). However, circulating PAG levels was seen to vary among sires of different fertility (FIG. 15). Likewise, embryonic loss varied among bulls (FIG. 16).

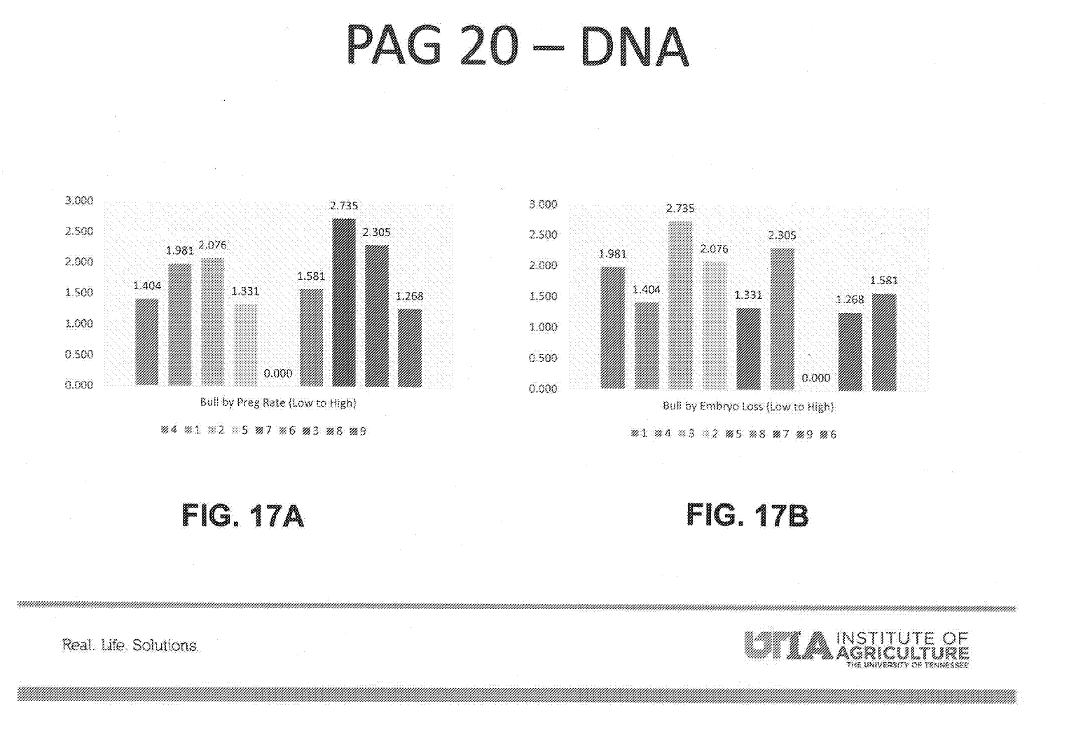

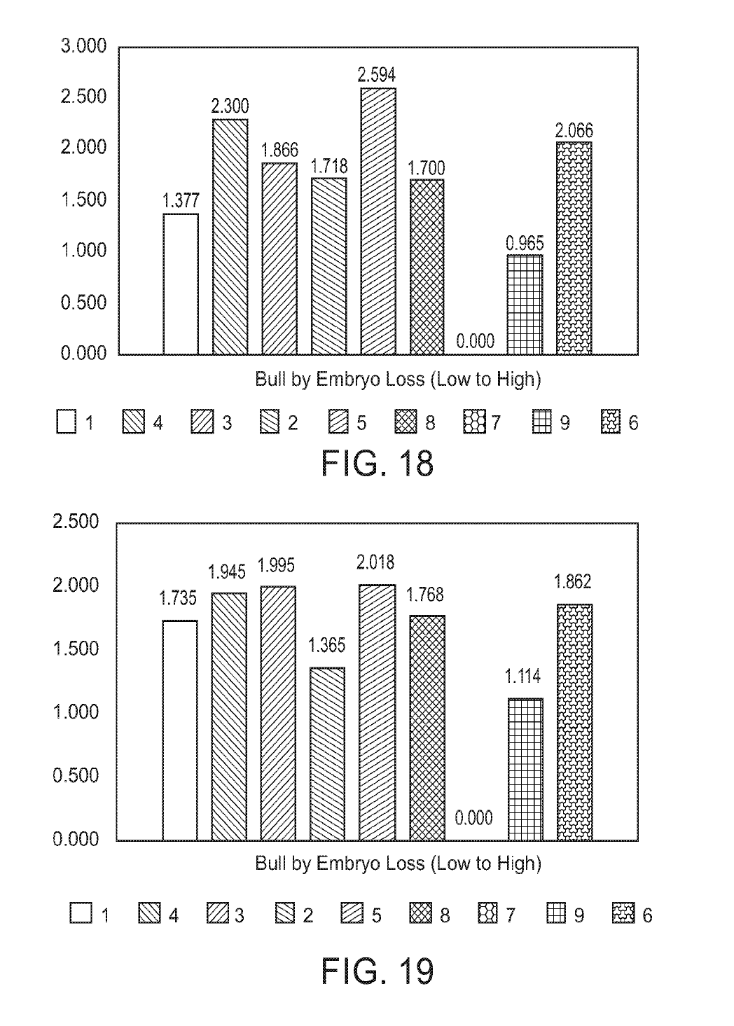

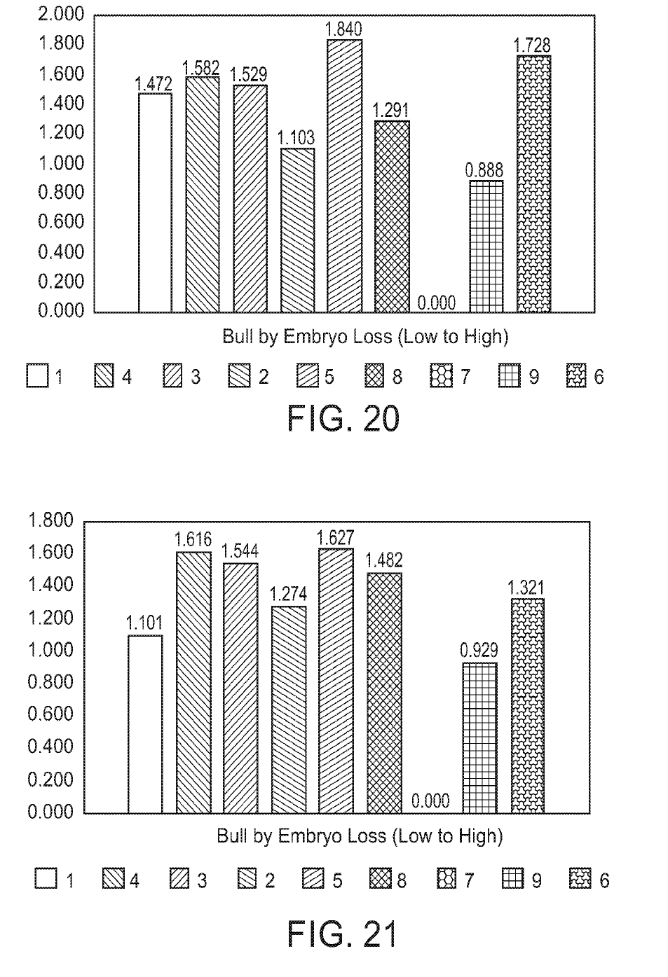

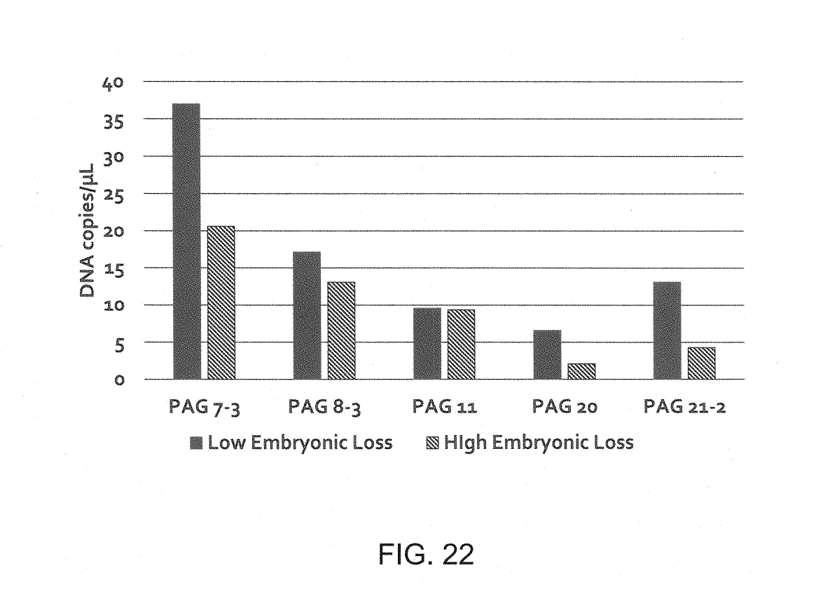

Considered by gene of interest, genomic PAG levels appeared related to embryo loss, i.e. lower genomic PAG levels appeared predictive of early embryo loss (see FIGS. 17B and 18-21) but not of pregnancy rate (FIG. 17A).

Summarizing, from the present results bulls exhibiting a high pregnancy loss between days 29 to term of pregnancy also show decreased circulating concentrations of PAG protein. Likewise, these bulls tend to have decreased relative abundance of the various PAG genes evaluated. As such, the tested PAG genes appear to be predictive of higher pregnancy loss, i.e. decreased calves born following insemination. Interestingly, however, the predictive nature of PAG gene quantification did not appear to extend to prediction of pregnancy rate.

The present disclosure thus responds to a need in the art by providing effective methods for identification of bulls exhibiting increased fertility, i.e. bulls less likely to result in an early embryonic death following insemination or other reproductive procedures such as in vitro fertilization. This could be a valuable finding in terms of selecting for bulls likely to result in a pregnancy extending to term rather than only an enhanced pregnancy rate.

While the terms used herein are believed to be well-understood by one of ordinary skill in the art, definitions are set forth to facilitate explanation of certain of the presently-disclosed subject matter.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter. With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, S, C, and/or O” includes A, S, C, and O individually, but also includes any and all combinations and subcombinations of A, S, C, and O.

The term “antibody” (Ab) as used herein includes monoclonal antibodies, polyclonal antibodies, multispecific antibodies and antibody fragments, as long as they exhibit the desired biological activity. The term “polyclonal antibody” as used herein refers to an antibody obtained from a population of heterogeneous antibodies, i.e., they are secreted by different B cell lineages within the body. The term “monoclonal antibody” as used herein refers to an antibody obtained from a population of substantially homogeneous antibodies, i.e., the individual antibodies that make up the population are identical except for possible naturally occurring mutations. Monoclonal antibodies are highly specific, being directed against a single antigenic site.

The term “antibody” (Ab) as used herein also includes antibody fragments. An “antibody fragment” is a portion of an intact antibody, preferably the antigen binding or variable region of the intact antibody. Examples of antibody fragments include but are not limited to: Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

The term “semen” as used herein refers to the fluid and sperm cells contained therein in mammals. Semen as used herein includes neat and diluted semen.

The foregoing description of preferred embodiments has been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the disclosed subject matter and its practical application to thereby enable one of ordinary skill in the art to utilize the invention in various embodiments and with various modifications as are suited to the particular use contemplated. All such modifications and variations are within the scope of the invention as determined by the claims when interpreted in accordance with the breadth to which they are fairly, legally and equitably entitled.

### REFERENCES

[1] Diskin M G, Morris D G. Embryonic and early foetal losses in cattle and other ruminants. Reproduction in domestic animals=Zuchthygiene. 2008; 43 Suppl 2:260-7.
[2] Ayalon N. A review of embryonic mortality in cattle. J Reprod Fertil. 1978; 54:483-93.
[3] Sreenan J, Diskin M. The extent and timing of embryonic mortality in the cow. Embryonic mortality in farm animals: Springer; 1986. p. 1-11.
[4] Cartmill J A, El-Zarkouny S Z, Hensley B A, Lamb G C, Stevenson J S. Stage of cycle, incidence, and timing of ovulation, and pregnancy rates in dairy cattle after three timed breeding protocols. J Dairy Sci. 2001; 84:1051-9.
[5] Cartmill J A, El-Zarkouny S Z, Hensley B A, Rozell T G, Smith J F, Stevenson J S. An alternative AI breeding protocol for dairy cows exposed to elevated ambient temperatures before or after calving or both. Journal of dairy science. 2001; 84:799-806.
[6] Lamb G C. Reproductive real-time ultrasound technology: An application for improving calf crop in cattle operations. In: Fields, M J, R S Sand, and JV Yelich (Eds), Factors Affecting Calf Crop: Biotechnology of Reproduction CRC Press, Boca Raton, Fla. 2002:235-53.
[7] Silke V, Diskin M G, Kenny D A, Boland M P, Dillon P, Mee J F, et al. Extent, pattern and factors associated with late embryonic loss in dairy cows. Anim Reprod Sci. 2002; 71:1-12.
[8] Stevenson J S, Lamb G C, Johnson S K, Medina-Britos M A, Grieger D M, Harmoney K R, et al. Supplemental norgestomet, progesterone, or melengestrol acetate increases pregnancy rates in suckled beef cows after timed inseminations. J Anim Sci. 2003; 81:571-86.
[9] Vasconcelos J L M, Silcox R W, Lacerda J A, Pursley J R, Wiltbank M C. Pregnancy rate, pregnancy loss, and response to heat stress after AI at 2 different times from ovulation in dairy cows. Biol Reprod. 1997; 56:140.
[10] Pereira M H, Cooke R F, Alfieri A A, Vasconcelos J L. Effects of vaccination against reproductive diseases on reproductive performance of lactating dairy cows submitted to AI. Anim Reprod Sci. 2013; 137:156-62.
[11] Aono F H, Cooke R F, Alfieri A A, Vasconcelos J L. Effects of vaccination against reproductive diseases on reproductive performance of beef cows submitted to fixed-timed AI in Brazilian cow-calf operations. Theriogenology. 2013; 79:242-8.
[12] Spencer T E, Johnson G A, Bazer F W, Burghardt R C. Fetal-maternal interactions during the establishment of pregnancy in ruminants. Soc Reprod Fertil Suppl. 2007; 64:379-96.
[13] Spencer T E, Johnson G A, Bazer F W, Burghardt R C, Palmarini M. Pregnancy recognition and conceptus implantation in domestic ruminants: roles of progesterone, interferons and endogenous retroviruses. Reproduction, Fertility and Development. 2006; 19:65-78.
[14] Spencer T E, Sandra O, Wolf E. Genes involved in conceptus-endometrial interactions in ruminants: insights from reductionism and thoughts on holistic approaches. Reproduction. 2008; 135:165-79.
[15] Pohler K G, Geary T W, Atkins J A, Perry G A, Jinks E M, Smith M F. Follicular determinants of pregnancy establishment and maintenance. Cell Tissue Res. 2012; 349:649-64.
[16] Pereira M H, Wiltbank M C, Vasconcelos J L. Expression of estrus improves fertility and decreases pregnancy losses in lactating dairy cows that receive AI or embryo transfer using only cows that ovulate following the synchronization protocol. Journal of dairy science. 2015; Submitted.
[17] Breukelman S P, Perenyi Z, Taverne M A, Jonker H, van der Weijden G C, Vos P L, et al. Characterisation of pregnancy losses after embryo transfer by measuring plasma progesterone and bovine pregnancy-associated glycoprotein-1 concentrations. Vet J. 2012; 194:71-6.
[18] Perry G A, Smith M F, Lucy M C, Green J A, Parks T E, MacNeil M D, et al. Relationship between follicle size at insemination and pregnancy success. P Natl Acad Sci USA. 2005; 102:5268-73.
[19] Pohler K G, Geary T W, Johnson C L, Atkins J A, Jinks E M, Busch D C, et al. Circulating bovine pregnancy associated glycoproteins are associated with late embryonic/fetal survival but not ovulatory follicle size in suckled beef cows. J Anim Sci. 2013; 91:4158-67.
[20] Thompson I M, Cerri R L, Kim I H, Green J A, Santos J E, Thatcher W W. Effects of resynchronization programs on pregnancy per artificial insemination, progesterone, and pregnancy-associated glycoproteins in plasma of lactating dairy cows. J Dairy Sci. 2010; 93:4006-18.
[21] Bellow R, Staigmiller R, Wilson J, Phelps D, Darling A. Use of bovine FSH for superovulation and embryo production in beef heifers. Theriogenology. 1991; 35:1069-82.
[22] Kirby C J, Smith M F, Keisler D H, Lucy M C. Follicular function in lactating dairy cows treated with sustained-release bovine somatotropin. J Dairy Sci. 1997; 80:273-85.
[23] Green J A, Parks T E, Avalle M P, Telugu B P, McLain A L, Peterson A J, et al. The establishment of an ELISA for the detection of pregnancy-associated glycoproteins (PAGs) in the serum of pregnant cows and heifers. Theriogenology. 2005; 63:1481-503.
[24] Mercadante P M, Waters K M, Mercadante V R, Lamb G C, Elzo M A, Johnson S E, et al. Subspecies differences in early fetal development and plasma pregnancy-associated glycoprotein concentrations in cattle. J Anim Sci. 2013; 91:3693-701.
[25] Robbins K R, Saxton A M, Southern L L. Estimation of nutrient requirements using broken-line regression analysis. J Anim Sci. 2006; 84 Suppl:E155-65.
[26] Butler J E, Hamilton W C, Sasser R G, Ruder C A, Hass G M, Williams R J. Detection and partial characterization of two bovine pregnancy-specific proteins. Biol Reprod. 1982; 26:925-33.
[27] Mialon M M, Camous S, Renand G, Martal J, Menissier F. Peripheral concentrations of a 60-kDa pregnancy serum protein during gestation and after calving and in relationship to embryonic mortality in cattle. Reprod Nutr Dev. 1993; 33:269-82.
[28] Sasser R G, Ruder C A, Ivani K A, Butler J E, Hamilton W C. Detection of pregnancy by radioimmunoassay of a novel pregnancy-specific protein in serum of cows and a profile of serum concentrations during gestation. Biol Reprod. 1986; 35:936-42.
[29] Zoli A P, Beckers J F, Wouters-Ballman P, Closset J, Falmagne P, Ectors F. Purification and characterization of a bovine pregnancy-associated glycoprotein. Biol Reprod. 1991; 45:1-10.
[30] Zoli A P, Guilbault L A, Delahaut P, Ortiz W B, Beckers J F. Radioimmunoassay of a bovine pregnancy-associated glycoprotein in serum: its application for pregnancy diagnosis. Biol Reprod. 1992; 46:83-92.
[31] Leblanc S J. Short communication: field evaluation of a pregnancy confirmation test using milk samples in dairy cows. J Dairy Sci. 2013; 96:2345-8.
[32] Kill L K, Pohler K G, Perry G A, Smith M F. Serum bovine pregnancy associated glycoproteins and progesterone in beef heifers that experienced late embryonic/fetal mortality. J Anim Sci Midwest Meetings. 2013.
[33] Humblot P. Use of pregnancy specific proteins and progesterone assays to monitor pregnancy and determine the timing, frequencies and sources of embryonic mortality in ruminants. Theriogenology. 2001; 56:1417-33.
[34] Pohler K G, Pereira M H, Lopes F R, Lawrence J C, Keisler D H, Smith M F, et al. Circulating concentrations of bovine pregnancy associated glycoproteins and late embryonic mortality in lactating dairy herds. Journal of dairy science. 2015:Accepted.
[35] Ricci A, Carvalho P D, Amundson M C, Fourdraine R H, Vincenti L, Fricke P M. Factors associated with pregnancy-associated glycoprotein (PAG) levels in plasma and milk of Holstein cows during early pregnancy and their effect on the accuracy of pregnancy diagnosis. J Dairy Sci. 2015; 98:2502-14.
[36] Humblot F, Camous S, Martal J, Charlery J, Jeanguyot N, Thibier M, et al. Pregnancy-specific protein B, progesterone concentrations and embryonic mortality during early pregnancy in dairy cows. J Reprod Fertil. 1988; 83:215-23.
[37] Wallace J M, Aitken R P, Cheyne M A, Humblot P. Pregnancy-specific protein B and progesterone concentrations in relation to nutritional regimen, placental mass and pregnancy outcome in growing adolescent ewes carrying singleton fetuses. J Reprod Fertil. 1997; 109:53-8.
[38] Lobago F, Bekana M, Gustafsson H, Beckers J F, Yohannes G, Aster Y, et al. Serum profiles of pregnancy-associated glycoprotein, oestrone sulphate and progesterone during gestation and some factors influencing the profiles in Ethiopian Borana and crossbred cattle. Reproduction in domestic animals=Zuchthygiene. 2009; 44:685-92.
[39] Patel O V, Sulon J, Beckers J F, Takahashi T, Hirako M, Sasaki N, et al. Plasma bovine pregnancy-associated glycoprotein concentrations throughout gestation in relationship to fetal number in the cow. Eur J Endocrinol. 1997; 137:423-8.
[40] Wallace R M, Pohler K G, Smith M F, Green J A. Placental PAGs: gene origins, expression patterns, and use as markers of pregnancy. Reproduction. 2015; 149:R115-26.

1. A method for selecting for enhanced fertility of a male mammal, comprising:
obtaining one or more biological samples from a plurality of male mammals;
quantifying a pregnancy-associated glycoprotein (PAG) genomic DNA in the one or more biological samples; and
selecting one or more male mammals of the plurality of male mammals exhibiting a highest PAG genomic DNA.

2. The method of claim 1, further including using a cell or tissue obtained from the selected one or more male mammals exhibiting the highest PAG genomic DNA in a reproductive procedure selected from the group consisting of one or more of a natural insemination procedure, an artificial insemination procedure, an in vitro fertilization procedure, and a cloning procedure.

3. The method of claim 1, wherein the plurality of male mammals are bovine animals.

4. (canceled)

5. (canceled)

6. (canceled)

7. The method of claim 1, wherein the step of quantifying the PAG genomic DNA includes quantifying a number of copies of one or more PAG genes selected from the group consisting of a PAG 7 gene, a PAG 8 gene, a PAG 11 gene, a PAG 20 gene, and a PAG 21 gene.

8. The method of claim 7, wherein:
the PAG 7 gene comprises one or both of SEQ ID NO: 15 and SEQ ID NO: 16;
the PAG 8 gene comprises one or more of SEQ ID NO: 17, SEQ ID NO: 18, SEQ ID NO: 19, and SEQ ID NO: 20;
the PAG 11 gene comprises one or both of SEQ ID NO: 21 and SEQ ID NO: 22;
the PAG 20 gene comprises one or both of SEQ ID NO: 23 and SEQ ID NO: 24; and
the PAG 21 gene comprises one or more of SEQ ID NO: 25, SEQ ID NO: 26, SEQ ID NO: 27, and SEQ ID NO: 28.

9. (canceled)

10. (canceled)

11. (canceled)

12. (canceled)

13. The method of claim 8, including:
quantifying the PAG 7 gene in genomic DNA by PCR using primers selected from one or both of SEQ ID NO: 1 and SEQ ID NO: 2;
quantifying the PAG 8 gene in genomic DNA by PCR using one or more primers selected from SEQ ID NO: 3, SEQ ID NO: 4, SEQ ID NO: 5, and SEQ ID NO: 6;
quantifying the PAG 11 gene in genomic DNA by PCR using primers selected from one or both of SEQ ID NO: 7 and SEQ ID NO: 8;
quantifying the PAG 20 gene in genomic DNA by PCR using primers selected from one or both of SEQ ID NO: 9 and SEQ ID NO: 10; and
quantifying the PAG 21 gene in genomic DNA by PCR using primers selected from one or more of SEQ ID NO: 11, SEQ ID NO: 12, SEQ ID NO: 13, and SEQ ID NO: 14.

14. (canceled)

15. (canceled)

16. (canceled)

17. (canceled)

18. A kit for determining enhanced fertility of a male mammal, comprising reagents for quantifying a pregnancy-associated glycoprotein (PAG) genomic DNA in one or more biological samples obtained from a plurality of male mammals; and
optionally, one or more of: equipment for obtaining the one or more biological samples, additional reagents, and instructions for use of said reagents for quantifying.

19. The kit of claim 18, wherein the plurality of male mammals are bovine animals.

20. (canceled)

21. The kit of claim 18, including reagents for quantifying PAG genes selected from one or more of a PAG 7 gene, a PAG 8 gene, a PAG 11 gene, a PAG 20 gene, and a PAG 21 gene.

22. The kit of claim 21, wherein:
the PAG 7 gene comprises one or both of SEQ ID NO: 15 and SEQ ID NO: 16;
the PAG 8 gene comprises one or more of SEQ ID NO: 17, SEQ ID NO: 18, SEQ ID NO: 19, and SEQ ID NO: 20;
the PAG 11 gene comprises one or both of SEQ ID NO: 21 and SEQ ID NO: 22;
the PAG 20 gene comprises one or both of SEQ ID NO: 23 and SEQ ID NO: 24; and
the PAG 21 gene comprises one or more of SEQ ID NO: 25, SEQ ID NO: 26, SEQ ID NO: 27, and SEQ ID NO: 28.

23. (canceled)

24. (canceled)

25. (canceled)

26. (canceled)

27. The kit of claim 22, including primers adapted for quantifying a number of copies of PAG genes in genomic DNA by PCR and selected from the group consisting of:
one or both of SEQ ID NO: 1 and SEQ ID NO: 2 for quantifying the PAG 7 gene;
one or more of SEQ ID NO: 3, SEQ ID NO: 4, SEQ ID NO: 5, and SEQ ID NO: 6 for quantifying the PAG 8 gene;
one or both of SEQ ID NO: 7 and SEQ ID NO: 8 for quantifying the PAG 11 gene;
one or both of SEQ ID NO: 9 and SEQ ID NO: 10 for quantifying the PAG 20 gene; and
one or more of SEQ ID NO: 11, SEQ ID NO: 12, SEQ ID NO: 13, and SEQ ID NO: 14 for quantifying the PAG 21 gene.

28. (canceled)

29. (canceled)

30. (canceled)

31. (canceled)

32. A method for selecting for improved embryonic survival rate, comprising:
obtaining one or more samples of a cell or tissue from a plurality of bovine sires;
quantifying a paternal pregnancy-associated glycoprotein (PAG) genomic DNA in the one or more samples of a cell or tissue;
selecting one or more bovine sires of the plurality exhibiting a highest PAG genomic DNA; and
using a cell or tissue obtained from the selected one or more bovine sires exhibiting the highest PAG genomic DNA in a reproductive procedure selected from the group consisting of one or more of a natural insemination procedure, an artificial insemination procedure, an in vitro fertilization procedure, and a cloning procedure.

33. The method of claim 32, wherein the step of quantifying the paternal PAG genomic DNA includes quantifying a number of copies of one or more PAG genes selected from the group consisting of a PAG 7 gene, a PAG 8 gene, a PAG 11 gene, a PAG 20 gene, and a PAG 21 gene.

34. The method of claim 33, wherein:
the PAG 7 gene comprises one or both of SEQ ID NO: 15 and SEQ ID NO: 16;
the PAG 8 gene comprises one or more of SEQ ID NO: 17, SEQ ID NO: 18, SEQ ID NO: 19, and SEQ ID NO: 20;
the PAG 11 gene comprises one or both of SEQ ID NO: 21 and SEQ ID NO: 22;
the PAG 20 gene comprises one or both of SEQ ID NO: 23 and SEQ ID NO: 24; and
the PAG 21 gene comprises one or more of SEQ ID NO: 25, SEQ ID NO: 26, SEQ ID NO: 27, and SEQ ID NO: 28.

35. The method of claim 34, including:
quantifying the PAG 7 gene in genomic DNA by PCR using primers selected from one or both of SEQ ID NO: 1 and SEQ ID NO: 2;
quantifying the PAG 8 gene in genomic DNA by PCR using one or more primers selected from SEQ ID NO: 3, SEQ ID NO: 4, SEQ ID NO: 5, and SEQ ID NO: 6;
quantifying the PAG 11 gene in genomic DNA by PCR using primers selected from one or both of SEQ ID NO: 7 and SEQ ID NO: 8;
quantifying the PAG 20 gene in genomic DNA by PCR using primers selected from one or both of SEQ ID NO: 9 and SEQ ID NO: 10; and
quantifying the PAG 21 gene in genomic DNA by PCR using primers selected from one or more of SEQ ID NO: 11, SEQ ID NO: 12, SEQ ID NO: 13, and SEQ ID NO: 14.


SEQUENCE LISTING (40 sequences)

SEQ ID NO: 1 - DNA, 21 bp, artificial sequence
  PAG 7-3 Forward Primer (5′ - 3′)
gccactgctataccaccttta

SEQ ID NO: 2 - DNA, 24 bp, artificial sequence
  PAG 7-3 Reverse Primer (5′ - 3′)
ccttaatgattcttgagccagacc

SEQ ID NO: 3 - DNA, 20 bp, artificial sequence
  PAG 8-2 Forward Primer (5′ - 3′)
agacctggatcctgggtgat

SEQ ID NO: 4 - DNA, 20 bp, artificial sequence
  PAG 8-2 Reverse Primer (5′ - 3′)
tgtgtgtttgtctggccctg

SEQ ID NO: 5 - DNA, 20 bp, artificial sequence
  PAG 8-3 Forward Primer (5′ - 3′)
tttcaagggggcacagagat

SEQ ID NO: 6 - DNA, 21 bp, artificial sequence
  PAG 8-3 Reverse Primer (5′ - 3′)
attgattcctgtagcagcccg

SEQ ID NO: 7 - DNA, 20 bp, artificial sequence
  PAG 11 Forward Primer (5′ - 3′)
agtgattggctgtgaacacg

SEQ ID NO: 8 - DNA, 20 bp, artificial sequence
  PAG 11 Reverse Primer (5′ - 3′)
cacgaggcactgagtaatcg

SEQ ID NO: 9 - DNA, 20 bp, artificial sequence
  PAG 20 Forward Primer (5′ - 3′)
ccgggtcatcaaatatccaa

SEQ ID NO: 10 - DNA, 20 bp, artificial sequence
  PAG 20 Reverse Primer (5′ - 3′)
gacaacagagggcagagcac

SEQ ID NO: 11 - DNA, 23 bp, artificial sequence
  PAG 21-2 Forward Primer (5′ - 3′)
tataccgcctttaaaaagcaacg

SEQ ID NO: 12 - DNA, 21 bp, artificial sequence
  PAG 21-2 Reverse Primer (5′ - 3′)
gtgccctagtgtgagtgagtg

SEQ ID NO: 13 - DNA, 23 bp, artificial sequence
  PAG 21-3 Forward Primer (5′ - 3′)
gtgtcagaatacgggtttaagga

SEQ ID NO: 14 - DNA, 21 bp, artificial sequence
  PAG 21-3 Reverse Primer (5′ - 3′)
gatggggatggcttcagagaa

SEQ ID NO: 15 - DNA, 142 bp, Bos taurus
gtarargaggagatctaagagagctgggtcctgggtgaagtcttcctgaggctgtatttctcagtctttgatcgaggaaatgacaggattggcctggcacgggcagtgtaaatgcttggtctggctcaagaatcattaagga

SEQ ID NO: 16 - DNA, 143 bp, Bos taurus
agactycctggcccgtgccaggccaatcctgtctttcctcgatcaaagactgagaaatacagcctcaggaagacttcacccaggacccagctctctgtagatctcctcactcttttctctttaaaggtggtatagcagtggca

SEQ ID NO: 17 - DNA, 109 bp, Bos taurus
ccycggwmckgwttmtmgttttgaccgaggaaataacaggattggcctggctcctgcagcataaattcgggctgctacaggaatcaatcagggccagacaaacacacaa

SEQ ID NO: 18 - DNA, 109 bp, Bos taurus
gtkrykmgcccgaatttatgctgcaggagccaggccaatcctgttatttcctcggtcataaactgaaaaatacagcctcaggaagacatcacccaggatccagatctmg

SEQ ID NO: 19 - DNA, 122 bp, Bos taurus
acctcggagacctggatcctgggtgatgtcttcctgaggctgtatttttcagtttatgaccgaggaaataacaggattggcctggctcctgcagcataaattcgggctgctacaggaatcaa

SEQ ID NO: 20 - DNA, 134 bp, Bos taurus
acgckgcaggagccaggccaatcctgttatttcctcggtcataaactgaaaaatacagcctcaggaagacatcacccaggatccaggtctccgaggttcccaaatttttcaagatctctgtgcccccttgaaaa

SEQ ID NO: 21 - DNA, 264 bp, Bos taurus
gactacttgtggataccgggacatcactgatccatggcccagcaagaccagtcaccaacatccaaaagttcatccacgctatgccctacggttccgaggtgaagggtcatgacacaggtctctcccagttcccctacacaaggatctcctgggccaacctctctccctctttccccaacagtacatggttttgtgtcctgtcatcagtatcctgcctcctgtcatcttcaccatcaatggcatcgattactcagtgcctcgtga

SEQ ID NO: 22 - DNA, 264 bp, Bos taurus
cctawakgckagatgacaggaggcaggatactgatgacaggacacaaaaccatgtactgttggggaaagagggagagaggttggcccaggagatccttgtgtaggggaactgggagagacctgtgtcatgacccttcacctcggaaccgtagggcatagcgtggatgaacttttggatgttggtgactggtcttgctgggccatggatcagtgatgtcccggtatccacaagagcctcgcagccgtgttcacagccaatcacta

SEQ ID NO: 23 - DNA, 183 bp, Bos taurus
tgatcgataacatacacaagctcattggtgccacaccacggggttccaaggtgaagggtcatgccccagggtcactcccagtctccacacaaaacaaggatctccatggccaatctctctccctctctctctaacagcactacgtttcatgttctgcggtcagtgctctgccctctgttgtca

SEQ ID NO: 24 - DNA, 189 bp, Bos taurus
cgggatgaaacgtagtgctgttagagagagagggagagagattggccatggagatccttgttttgtgtggagactgggagtgaccctggggcatgacccttcaccttggaaccccgtggtgtggcaccaatgagcttgtgtatgttatcgatcactcttcctgggccttggatatttgatgacccrgra

SEQ ID NO: 25 - DNA, 153 bp, Bos taurus
gacggtctmgagactggctctgggtgcgccttcctgaggkgmktsmggcwtyagcwttmckcgakaaagamtgkgaaaarccctysrgaaggkgcaaccaggarccaggtctctgtaratgaamtgaawycttyyttttaarrggggtaaatc

SEQ ID NO: 26 - DNA, 167 bp, Bos taurus
gcggtarcggctactgattctgacactccaagcatttacactgcctgtgccaggccaatcctgccatttcctcgatcaaagacygagaaatacaccctcaggaaggcgtcacccaggagccaggtctctgtagatgaactgaatcgttgctttttaaaggcggtata

SEQ ID NO: 27 - DNA, 58 bp, Bos taurus
tgatggcatcctgggcttgaactaccccgacgaatccttctctgaagccatccccatc

SEQ ID NO: 28 - DNA, 64 bp, Bos taurus
gtacggggtagttcagcccaggatgccatcataagctctgtccttaacccgtattctgacatag

SEQ ID NO: 29 - DNA, 20 bp, artificial sequence
  primer
ctagacccctcagatgcagg

SEQ ID NO: 30 - DNA, 20 bp, artificial sequence
  primer
tgggacttagcaaggggaag

SEQ ID NO: 31 - DNA, 20 bp, artificial sequence
  primer
tcacaaggagaggggaacac

SEQ ID NO: 32 - DNA, 20 bp, artificial sequence
  primer
cattatggtgggagcactgc

SEQ ID NO: 33 - DNA, 20 bp, artificial sequence
  primer
tgagctaccccaacaaaacc

SEQ ID NO: 34 - DNA, 20 bp, artificial sequence
  primer
ttcgtgcaactgaaagcatc

SEQ ID NO: 35 - DNA, 20 bp, artificial sequence
  primer
ggagtcagggagagggaaac

SEQ ID NO: 36 - DNA, 20 bp, artificial sequence
  primer
caggcgaggactaggttgag

SEQ ID NO: 37 - DNA, 20 bp, artificial sequence
  primer
gttacccaaccaagccagaa

SEQ ID NO: 38 - DNA, 20 bp, artificial sequence
  primer
gagtggaatttcccgtgcta

SEQ ID NO: 39 - DNA, 19 bp, artificial sequence
  primer
tgcagagcaactgtgaatc

SEQ ID NO: 40 - DNA, 20 bp, artificial sequence
  primer
gcatgggcattcttgaagtt